(19) United States
(12) Patent Application Publication
(10) Pub. No.: US 2015/0148616 A1
(43) Pub. Date: May 28, 2015

(54) SYSTEMS AND METHODS FOR PROBABILITY BASED RISK PREDICTION

(71) Applicants: Washington State University, Pullman, WA (US); FedEx Corporation, Memphis, TN (US)

(72) Inventors: Hans P.A. Van Dongen, Spokane, WA (US); Suresh Rangan, Germantown, TN (US); Richard A. Lewis, Olive Branch, MS (US)

(21) Appl. No.: 14/554,891

(22) Filed: Nov. 26, 2014

Related U.S. Application Data
(60) Provisional application No. 61/909,826, filed on Nov. 27, 2013.

(51) Int. Cl. A61B 5/00; A61B 5/18

(52) U.S. Cl. CPC A61B 5/7275; A61B 5/4809; A61B 5/18; A61B 5/7246; A61B 5/7264; A61B 5/4815
USPC 600/300

(57) ABSTRACT

Systems and methods for probability based prediction of risks are disclosed herein. In one embodiment, a method includes measuring, using sensing elements individually associated with multiple users, a first set of data associated with the users. The method also includes transmitting to and storing in a computing device the measured first set of data and applying a performance model to the first set of data to generate a first set of performance values. The method further includes estimating a second set of data associated with the one or more users and applying the performance model to the second set of data to generate a second set of performance values. The method yet further includes determining if the first and second sets of performance values are statistically equivalent to each other.

### CROSS-REFERENCE TO RELATED APPLICATION(S)

This application claims priority to U.S. Provisional Application No. 61/909,826, filed on Nov. 27, 2013, the disclosure of which is incorporated herein by reference in its entirety.

### BACKGROUND

Fatigue from sleep loss, circadian misalignment, time on task, or other sources can degrade cognitive functioning and impairs performance, productivity, and safety. The personal, economic, and social costs involved in errors, incidents, and accidents resulting from fatigue are considerable in commercial aviation, trucking, mining, and other areas.

In Jan. 4, 2012, the Federal Aviation Administration (“FAA”) published flight and duty regulations on pilot scheduling for commercial flights to ensure pilots have adequate opportunity to rest before each duty day. The rules also allow airlines to develop alternative ways of mitigating pilot fatigue (i.e., alternative means of compliance) based on science and validated by sleep, performance, and safety data submitted to the FAA for approval. However, the rules create dilemmas for airlines attempting to develop an alternative means of compliance. On one hand, airlines cannot show compliance without collecting fatigue data when pilots operated according to the alternative means of compliance. On the other, airlines are not allowed to operate according to the alternative means of compliance without having the FAA's approval first based on the data.

### BRIEF DESCRIPTION OF THE DRAWINGS

FIG. 1 is a sleep homeostatic pressure versus time graph that shows an example study for fatigue distribution modeling in accordance with embodiments of the disclosed technology.

FIG. 2 is a schematic block diagram illustrating a fatigue prediction system in accordance with additional embodiments of the disclosed technology.

FIG. 3 is a flow diagram illustrating a process for probability based prediction of fatigue risk in accordance with embodiments of the disclosed technology.

FIG. 4 is a computing device suitable for certain components of the fatigue prediction system in FIG. 2.

### DETAILED DESCRIPTION

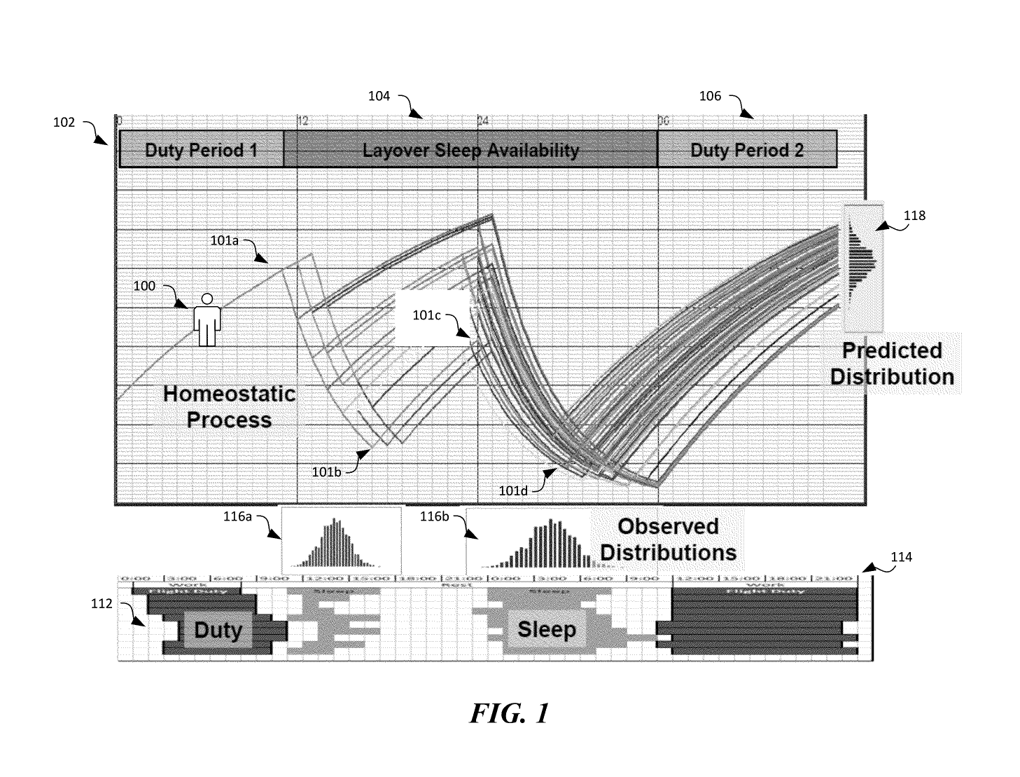

The present disclosure describes systems and methods for probability based prediction of fatigue risks. Based on such prediction, alternative means of compliance with scheduling rules promulgated by the FAA or other regulatory entities may be readily developed. Embodiments of the disclosed technology may also be used to compare fatigue distributions of two or more scheduling options for other reasons than (or in addition to) extant regulations. Even though particular systems, devices, components, and operations are disclosed in the following description, a person skilled in the relevant art will also understand that the technology may have additional embodiments, and that the technology may be practiced without several of the details of the embodiments described below with reference to FIGS. 1-4.

Fatigue resulting from sleep loss and circadian rhythm can be associated with decreased alertness reflected in reduced capacity to perform cognitive tasks and increased variability in performance. Thus, greater possibility of errors, incidents and accidents may result when pilots, drivers, or operators are fatigued. In around-the-clock operations, neurobiological mechanisms underlying fatigue mainly involve a sleep/wake homeostatic process and a circadian process. The sleep/wake homeostatic process tracks sleep history and seeks to balance time spent awake with an appropriate amount of recuperative sleep. The circadian process, driven by the biological clock in the brain, tracks time of day and seeks to place wakefulness during the day and sleep during the night.

The homeostatic and circadian processes normally operate in tandem to provide a stable level of alertness during the day and consolidated sleep during the night. However, when deviating from a normal schedule of daytime wakefulness and nighttime sleep, interaction of the two processes can lead to fatigue and associated decreased alertness and performance impairment. During nighttime operations, for example, the homeostatic and circadian processes align to steadily increase fatigue overnight, while also leading to difficulty to sleep during the day. When crossing time zones, the circadian process becomes temporarily desynchronized and may take several days or even weeks to adjust to the new time zone.

As used herein, the term “fatigue” generally refers to a level of deterioration in a person's alertness (as reflected in performance capability) as a function of sleep/wake history (time awake), circadian rhythm (time of day), sleep inertia (transient sleepiness immediately after awakening), workload (time on task, duty hours, nature of work), and/or other suitable factors. The experimentally determined effects of sleep/wake history and circadian rhythm on sleep propensity, alertness, and performance may be used to develop a mathematical model (referred herein as a “fatigue model”) for predicting fatigue based on such factors. One example can include a two-process model that invokes the homeostatic drive for sleep and the circadian rhythm in sleep propensity as processes driving sleepiness and fatigue. Other mathematical models can also use shift timing and duration (constituting a rough estimate of workload and/or time awake), as well as time of day (constituting a rough estimate of circadian rhythm phase) as their inputs.

With a fatigue model, it is believed that the impact of fatigue on a duty schedule can be evaluated without having to test that schedule in actual operation. As used herein, a “duty schedule” generally refers to a schedule according to which one or more individuals perform designated tasks, for example, flying a plane, driving a truck, operating machinery, etc. A fatigue model can be validated on specific data sets, and may then be generalized to predict the performance consequences of a potential schedule. The predictions of a fatigue model can be adjustable to predict objectively measurable loss in productivity (e.g., increased fuel consumption and/or increased maintenance in transportation) or other operationally relevant performance outcomes. One suitable fatigue model is disclosed in a Publication by Gregory D. Roach et al., entitled “A MODEL TO PREDICT WORK-RELATED FATIGUE BASED ON HOURS OF WORK,” published in Aviation, Space, and Environmental Medicine, Vol. 75, No. 3, Section II, March 2004, the disclosure of which is incorporated herein in its entirety.

The inventors have recognized that sleep timing and duration not only depend on biological processes (e.g., homeostatic and circadian processes) but also on non-biological factors, especially when individuals work consecutive nights and/or traversing multiple time zones. Aside from the sleep/wake/work schedule itself, these factors range from availability of hotel facilities (check-in/out times) or store opening hours to communications with family at home, etc. Some of these factors vary substantially from person to person and from duty to duty, such that pre-duty, in-flight, layover, and post-duty sleep schedules exhibit probabilistic distributions. A sleep prediction model for sleep timing and duration can be developed based not just on the underlying biological processes but also on systematic patterns in observations of real-world in-flight and layover sleep behaviors, as a function of prior and planned duty schedule, time of day, and/or location. The development of the sleep prediction model allows for fatigue distribution modeling by making probabilistic predictions of alertness that account for components of natural variability in sleep behavior.

FIG. 1 is a sleep homeostatic pressure versus time graph that shows an example study for fatigue distribution modeling in accordance with embodiments of the disclosed technology. Data shown in FIG. 1 were obtained from the study using wrist-worn rest/activity monitors in eleven pilots for a simulated forty eight hour duty schedule. The schedule involved a nine hour duty period starting at 01:00 AM followed by a twenty five hour layover period. A second twelve hour duty period starting at noon followed the layover period. Alertness predictions were made across the forty eight hour scenario using a two-process model. FIG. 1 only shows the homeostatic sleep pressure component of the alertness predictions. The circadian rhythm component of the alertness predictions is not shown in FIG. 1 for clarity purposes.

As shown in FIG. 1, a first duty period 102 is followed by a layover period 104 with sleep availability. A second duty period 106 then follows the layover period 104. During the first and second duty periods 102 and 106, the individuals had duty schedules 112 and 114 that have varying duty periods. In the illustrated embodiment, the duty periods in each of the duty schedules 112 and 114 overlap with one another. In other embodiments, at least one of the duty periods in each of the duty schedules 112 and 114 may not overlap with others.

FIG. 1 also shows homeostatic sleep pressure values of the individuals as a function of time observed during the study. Each line in FIG. 1 represents a homeostatic value for one individual. The homeostatic value increases when an individual was awake and decreases when the individual was asleep. For example, as shown in FIG. 1, an individual 100 had a homeostatic value that increases during the first duty period 102 until a time point 101a when the layover period 104 starts. The homeostatic value of the individual 100 then decreased from the time point 101a to a time point 101b by sleeping during this period. The individual 100 then woke up after the time point 101b and stayed awake until a time point 101c when the individual 100 went back to sleep again until a time point 101d. Even though the individual 100 could sleep from the time point 101a to the time point 101d, the individual 100 had two sleeping periods (i.e., between the first and second time points 101a and 101b and between the third and fourth time points 101c and 101d) before the second duty period 106 starts. The other individuals also exhibited similar behaviors (but not exactly the same) as reflected in the two distributed sleep patterns 116a and 116b.

Based on the observed sleep pattern distribution, fatigue (or a reduction of alertness) distribution 118 can be predicted for the second duty period 106. In the study, at the end of the second duty period 106, alertness scores of the pilots were 0.35±0.05 (mean±standard deviation). Forty eight hours earlier, the alertness score of the same pilots was 0.67. Such alertness distribution information can be used to statistically compare the predictions to those of other possible duty schedules, and to make informed decisions regarding the need for alertness-enhancing countermeasures.

Several embodiments of the disclosed technology are directed to probability based prediction of fatigue risks utilizing a fatigue model. In certain embodiments, a first set of data representing sleep patterns of one or more individuals (e.g., pilots, drivers, operator, etc.) operating under a first duty schedule are measured. A second set of data representing sleep patterns of the individuals operating under a second duty schedule can be estimated based on biology, experiments, measured data from other operation areas, and/or other suitable information. The fatigue model can then be applied to both the first and second sets of data representing sleep patterns to generate first and second distributions of fatigue levels (or alertness) of the individuals. Statistical analysis can then be applied to the first and second distributions of fatigue levels (or alertness) to measure a probability of equivalency (or non-inferiority) between the first and second duty schedules. As such, airlines, trucking companies, or other suitable entities may develop alternative means of compliance with governmental scheduling rules without the need to actually operate under proposed alternative schedules, or may develop alternative schedules that are less fatiguing, more productive, and/or safer than existing schedules.

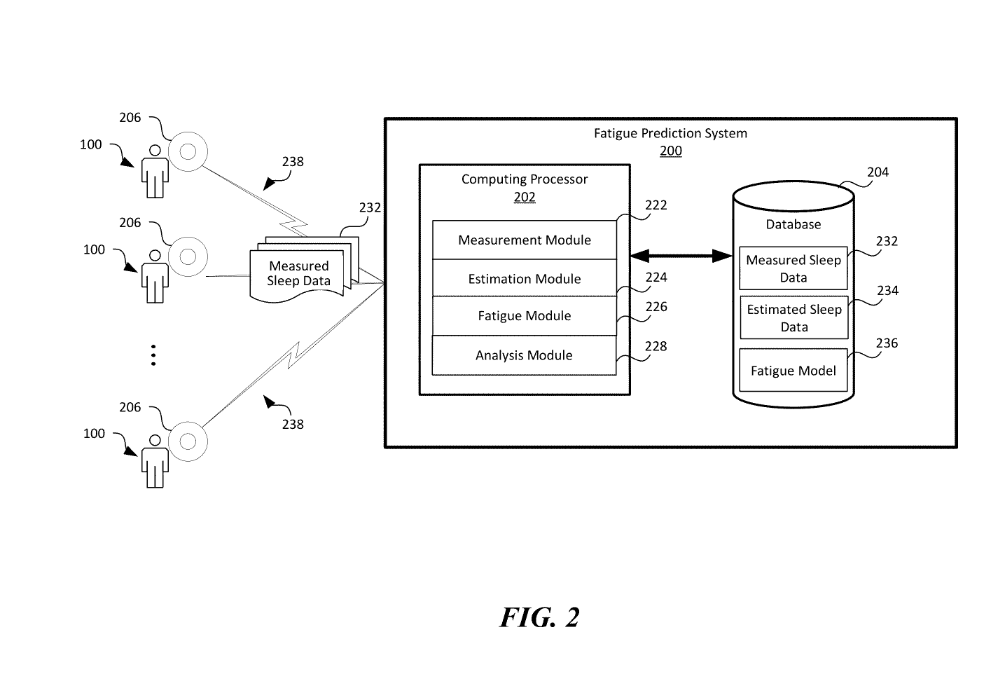

FIG. 2 is schematic block diagrams illustrating probability based prediction of fatigue risks in accordance with embodiments of the disclosed technology. In FIG. 2 and in other Figures herein, individual software modules, components, and routines may be a computer program, procedure, or process written as source code in C, C#, C++, Java, and/or other suitable programming languages. The computer programs, procedures, or processes may be compiled into intermediate, object, or machine code and presented for execution by a processor of a personal computer, a network server, a laptop computer, a server computer, or other suitable computing devices. Various implementations of the source, intermediate, and/or object code and associated data may be stored in one or more computer readable storage media that include read-only memory, random-access memory, magnetic disk storage media, optical storage media, flash memory devices, and/or other suitable media. As used herein, the term “computer readable storage media” excludes propagated signals, per se.

As shown in FIG. 2, the fatigue prediction system can include a computing processor 202 operatively coupled to a database 204. The computing processor 202 can include a micro-processor, a field programmable gate array, and/or other suitable processing elements. The database 204 can contain records of measured sleep data 232, estimated sleep data 234, and one or more fatigue models 236. Examples of the measured and/or estimated sleep data 232 and 234 can include at least one or more of sleep starting time, sleep ending time, time of day during sleep, and/or other suitable information. In certain embodiments, the individuals 100 can have a generally similar starting state or alertness level, for example, as shown in FIG. 1. In other embodiments, the individuals 100 can have distributed starting states. In the illustrated embodiment, the computing processor 202 and the database 204 are shown as being integrated into the fatigue prediction system 200. In other embodiments, the database 204 may be hosted by a remote server or a plurality of remote servers (not shown).

The computing processor 202 can be configured to execute instructions contained in a memory (not shown in FIG. 2) that provide a measurement module 222, an estimation module 224, a fatigue module, and an analysis module 228. Even though particular modules are show in FIG. 2, in other embodiments, the computing processor 202 may also include input modules (e.g., keyboard drivers), output modules (e.g., printer drivers), communications modules (e.g., network drivers), and/or other suitable types of modules. In further embodiments, various modules of the computing processor 202 may also be executed by additional and/or different computing processors (not shown).

The measurement module 222 is configured to obtain the measured sleep data 232 from one or more individuals 100 (e.g., pilots, drivers, machinery operators, etc.) operating according to a base duty schedule. In one embodiment, the base duty schedule can be a schedule approved by a governmental entity (e.g., FAA). In other embodiments, the base duty schedule can be an existing operating schedule or other suitable types of schedule. In the illustrated embodiment, each individual 100 carries or is otherwise associated with a sensing element 206 configured to detect and record measured sleep data 232. In one example, the sensing element 206 can include a portable rest/activity monitor that can be carried by the individuals 100. One suitable monitor (model No. GT9X ActiGraph Link) is provided by the ActiGraph Company of Pensacola, Fla. In other examples, the sensing element 206 can include a wrist actigraphy device, a fab, a smartphone, a laptop computer, and/or other suitable devices configured to record a sleep log from the individuals 100.

In certain embodiments, the sensing elements 206 may upload the recorded measured sleep data 232 to the measurement module 222 on a periodic, continuous, or other suitable basis via a computer network, a hardwire connection, a mobile phone network, and/or other suitable types of communications channel 238. In other embodiments, the measurement module 222 may poll the various sensing elements 206 on a periodic or other suitable basis. In yet other embodiments, the measurement module 222 can prompt the individuals 100 or other suitable operators to download the recorded measured sleep data 232 on a periodic or other suitable basis. In further embodiments, the sensing elements 206 may be omitted, and the individuals 100 may supply the measured sleep data 232 to the measurement module 222 by logging into a website (not shown) linked to the measurement module 222 and input the sleep data 232. In yet further embodiments, the measurement module 222 may also obtain the measured sleep data 232 using implantable devices (not shown), manual recording, or other suitable components and/or techniques.

The measurement module 222 is also configured to organize and store the measured sleep data 232 in the database 204. In one embodiment, the measurement module 222 can be configured to create records of the measured sleep data 232 corresponding to each of the individuals 100 based on at least one of an identification of the individual 100, a group the individual 100 belongs to, a work schedule, and/or other suitable criteria. In other embodiments, the measurement module 222 can also be configured to calculate certain sleep parameters based on the measured sleep data 232. For example, the measurement module 222 can be configured to calculate a duration of sleep, an average duration of sleep over a period of time, and/or other suitable parameters. In further embodiments, the measurement module 222 can be configured to manipulate and/or otherwise process the measured sleep data 232 before storing the measured sleep data 232 in the database 204.

The estimation module 224 is configured to generate a set of estimated sleep data 234 for the individuals 100 based on a test duty schedule different than the base duty schedule. In one embodiment, the test duty schedule can be a proposed duty schedule as an alternative means of compliance with a governmental regulation. In other embodiments, the test duty schedule may be a schedule under study or other suitable types of schedule. In certain embodiments, the estimation module 224 can be configured to obtain the estimated sleep data 234 based on circadian parameters, age, gender, and/or other suitable biological parameters of the individuals 100. In another embodiments, the estimation module 224 can generate the estimated sleep data 234 by observing and recording sleep data of the individuals 100 in an experimental setting without the individuals 100 actually performing the duties according to the test duty schedule.

In yet another embodiment, the estimation module 224 can generate the estimated sleep data 234 from areas of operation other than that associated with the test duty schedule and/or the individuals 100. For example, the estimated sleep data 234 for pilots may be generated based on sleep data for ground crews who operate according to a schedule generally similar to or the same as the test duty schedule. In a further embodiment, the estimation module 224 can also estimate the set of estimated sleep data 234 based on the measured sleep data 232 via interpolation, extrapolation, and/or other suitable techniques. In yet further embodiments, the estimated sleep data 234 can also be obtained from individuals 100 actually operating according to the test duty schedule, for example, because an exemption has been granted to collect data, or because the regulations are not yet applicable (e.g., before an effective date of the regulations). In the illustrated embodiment, the estimated sleep data 234 are stored in the database 204. In other embodiments, the estimated sleep data 234 may be stored in other suitable locations.

The fatigue module 226 is configured to apply the fatigue model 236 to the measured sleep data 232 and the estimated sleep data 234 to generate (1) a set of fatigue values corresponding to the base duty schedule (referred to as “base fatigue values”); and (2) a set of fatigue values corresponding to the test duty schedule (referred to as “test fatigue values”). In certain embodiments, the fatigue module 226 can be applied to the measured and estimated sleep data 232 and 234 on an individual basis. In other embodiments, the fatigue module 226 can be applied to permutations of the measured or estimated sleep data 232 and 234 by combining portions of the sleep data 232 and 234. For instance, one permutation of the measured sleep data 232 can include the first sleep period between the time points 101a and 101b of the first individual 100 and a second sleep period approximately between the time points 101c and 101d of a second individual 100 in FIG. 1. In such embodiments, random or non-random sampling from possible permutations may be used to limit a computational burden if a number of the permutations escalates. As discussed above, the measured sleep data 232 and the estimated sleep data 234 both include a distribution of values. As a result, the base fatigue values and the test fatigue values can both include a distribution of values after the fatigue model 236 is applied. As discussed in more detail below, the base and test fatigue values may then be analyzed by the analysis module 228 to determine if the test duty schedule carries a fatigue risk higher than, generally equivalent to, or lower than the base duty schedule.

The analysis module 228 is configured to apply statistical analysis or other suitable comparison technique (e.g. graphical, tabular, and/or visual comparison of distribution overlap) on the base and test fatigue values and can include various routines configured to calculate various statistical parameters. For example, in one embodiment, the analysis module 228 is configured to determine a mean, average, range, variability (e.g., standard deviation, interquartile range, median absolute deviation, etc.), or other suitable statistical parameters of the base and test fatigue values. The analysis module 228 can then compare the determined statistical parameters of the base and test fatigue values. In one example, the analysis module 228 can indicate that the base duty schedule carries a lower fatigue risk if at least one of the following statistical parameters associated with the test fatigue values are lower than corresponding ones associated with the base fatigue values:
mean fatigue value;
average fatigue value;
range of fatigue value;
standard deviation of fatigue values;
mean difference of fatigue values;
median absolute deviation of fatigue values;
average absolute deviation of fatigue values;
distance standard deviation of fatigue values.
   In certain embodiments, the foregoing comparison may be performed across the entire span of the base and test duty schedules, across selected portions of the foregoing duty schedules, or as associated with specific sections or events such as critical phases of a flight (e.g., take-offs and landings). In other embodiments, equivalence testing may be performed only on quantiles of the distributions that represent the greatest risk (e.g., the right-hand tails of the distributions). Such an approach may focus on the risks that may lead to the most catastrophic outcome rather than risks in general. In other examples, the foregoing determination may be based on other suitable statistical parameters or test statistics (e.g., t test statistics, F statistics, chi-square, etc.).

In operation, the measurement module 222 obtains the measured sleep data 232 from, for example, the sensing elements 206 associated with the individuals 100. The estimation module 224 can estimate or otherwise generate the estimated sleep data 234 based on the test duty schedule. In one embodiment, the test duty schedule can be input by an operator (not shown). In another embodiment, the test duty schedule can be generated by an application or process executed by the computing processor 202 or other processors (not shown). In yet further embodiments, the test duty schedule may be generated in other suitable manners.

The fatigue module 226 can then apply the fatigue model 236 to both the measured sleep data 232 and the estimated sleep data 234 to generate the base and test fatigue values, respectively. The analysis module 228 can then perform statistical analysis on the base and test fatigue values to determine if the test fatigue values are statistically superior than, equivalent to, or inferior than the base fatigue values. In response to determining that the test fatigue values are statistically superior than and/or equivalent to the base fatigue values, the analysis module 228 can indicate that the test duty schedule is at least equivalent, if not superior to, the base duty schedule.

In accordance with certain aspects of the disclosed technology, the test fatigue values are not generated based on data measured when the individuals 100 actually perform duties according to the test duty schedule. Instead, the test fatigue values are generated based on the estimated sleep data 232 without the need to actually having the individuals perform duties according to the test duty schedule. Thus, airlines may develop alternative means of compliance with the FAA's scheduling rules even without the FAA's prior approval for the test duty schedule for the individuals 100. As a result, sufficient savings in operating expenses as well as greater safety to pilots and the public may be achieved. In further embodiments, other suitable information (e.g., age, rank, experience, and/or genotype) may also be used to affect the predictions. The suitable information may also include actual fatigue data obtained during part of the schedule (e.g., the beginning part that may be flown without exceeding the applicable regulations). Such data may replace portions of the test fatigue values from the fatigue model predictions where applicable (e.g., to generate initial values) or be combined therewith using the Bayesian technique as disclosed in U.S. Pat. No. 8,781,796, the disclosure of which is incorporated herein in its entirety.

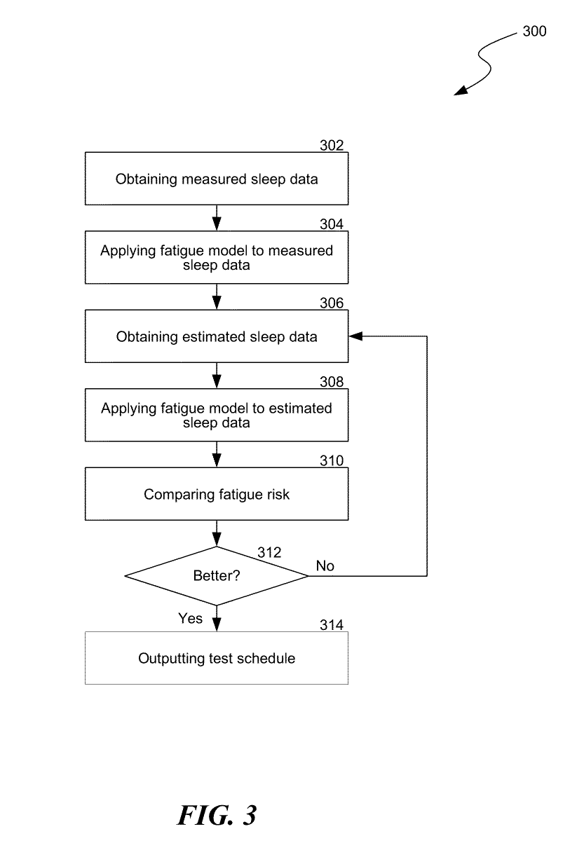

FIG. 3 is a flow diagram illustrating a process 300 for probability based prediction of fatigue risk in accordance with embodiments of the disclosed technology. As shown in FIG. 3, the process 300 includes obtaining measured sleep data of individuals operating according to a base duty schedule at stage 302. As discussed in more detail above with reference to FIG. 2, the measured sleep data may be obtained via the sensing elements 206 (FIG. 2) or via other suitable components. The process 300 can then include applying a fatigue model to the measured sleep data 304 to generate a set of base fatigue values at stage 304. In one embodiment, the fatigue model can be represented as a function of homeostatic and circadian parameters. Applying the fatigue model includes calculating a set of base fatigue values using the measured sleep data as input to the function. In other embodiments, the fatigue model may be represented as tables, graphs, and/or in other suitable manners. Applying the fatigue model can include table or graph lookups and/or other suitable operations.

The process 300 can also include obtaining estimated sleep data related to a test duty schedule for the individuals at stage 306. As discussed above with reference to FIG. 2, the estimated sleep data may be obtained in various fashions without the need to actually measure sleep data of the individual operating according to the test duty schedule. The process 300 can then include applying the same fatigue model to the estimated sleep data at stage 308 to generate a set of test fatigue values.

The process 300 can further include comparing the set of base fatigue values to the set of test fatigue values to determine a statistical relationship between the two sets of data at stage 310. Various suitable techniques for performing such statistical analysis are discussed above with reference to the analysis module 228 in FIG. 2. The process 300 can then include a decision stage 312 to determine if the test duty schedule is better than the base duty schedule based on the comparison of the base and test fatigue values. In one embodiment, the test duty schedule is indicated to be better than the base duty schedule if the test fatigue values are statistically equivalent or superior than the base fatigue values. In one example, the test duty schedule is statistically equivalent to the base duty schedule if a difference between corresponding statistical parameters (e.g., a mean value) of the base and test fatigue values is within a predetermined threshold. In other examples, the foregoing determination can also be based on other suitable criteria. In response to determining that the test duty schedule is better than the base duty schedule, the process 300 can include outputting the test duty schedule at stage 314; otherwise, the process 300 can revert to stage 306 based on another test duty schedule.

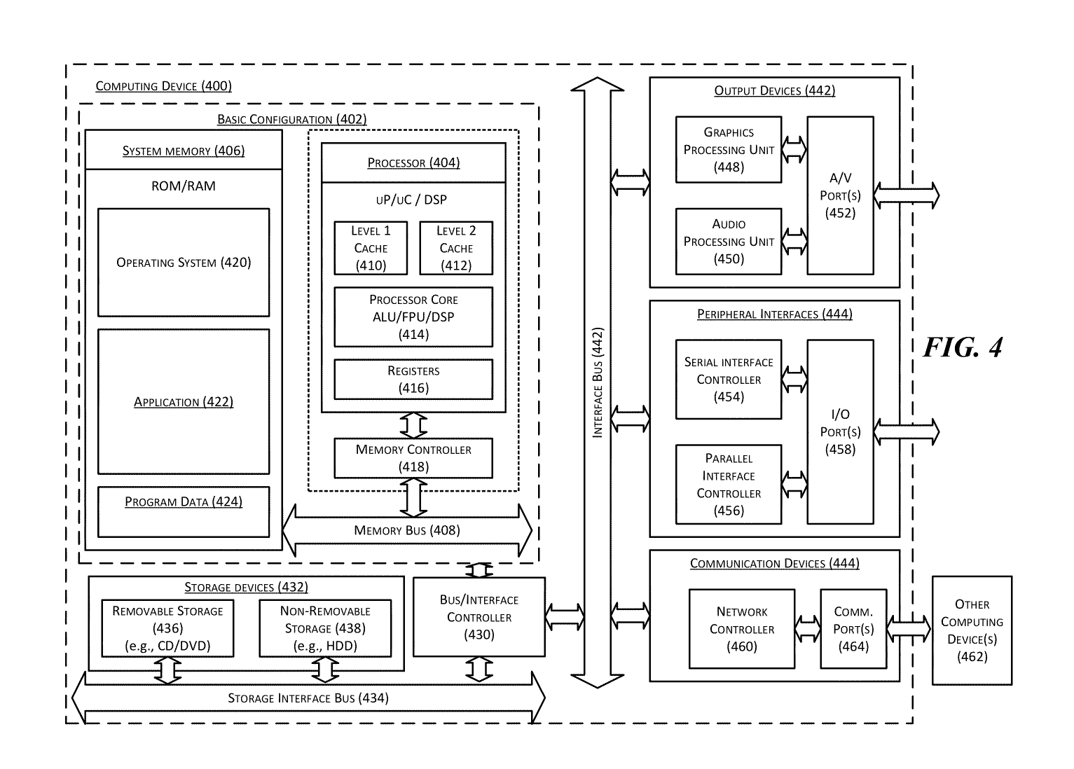

FIG. 4 is a computing device 400 suitable for the fatigue prediction system in FIG. 2. In a very basic configuration 402, computing device 400 typically includes one or more processors 404 and a system memory 406. A memory bus 408 may be used for communicating between processor 404 and system memory 406.

Depending on the desired configuration, the processor 404 may be of any type including but not limited to a microprocessor (μP), a microcontroller (μC), a digital signal processor (DSP), or any combination thereof. The processor 404 may include one more levels of caching, such as a level one cache 410 and a level two cache 412, a processor core 414, and registers 416. An example processor core 414 may include an arithmetic logic unit (ALU), a floating point unit (FPU), a digital signal processing core (DSP Coe), or any combination thereof. An example memory controller 418 may also be used with processor 404, or in some implementations memory controller 418 may be an internal part of processor 404.

Depending on the desired configuration, the system memory 406 may be of any type including but not limited to volatile memory (such as RAM), non-volatile memory (such as ROM, flash memory, etc.) or any combination thereof. The system memory 406 may include an operating system 420, one or more applications 422, and program data 424. This described basic configuration 402 is illustrated in FIG. 4 by those components within the inner dashed line.

The computing device 400 may have additional features or functionality, and additional interfaces to facilitate communications between basic configuration 402 and any other devices and interfaces. For example, a bus/interface controller 430 may be used to facilitate communications between the basic configuration 402 and one or more data storage devices 432 via a storage interface bus 434. The data storage devices 432 may be removable storage devices 436, non-removable storage devices 438, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

The system memory 406, removable storage devices 436 and non-removable storage devices 438 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 400. Any such computer storage media may be part of computing device 400. The term “computer storage medium” excludes propagated signals and communication media.

The computing device 400 may also include an interface bus 440 for facilitating communication from various interface devices (e.g., output devices 442, peripheral interfaces 444, and communication devices 446) to the basic configuration 402 via bus/interface controller 430. Example output devices 442 include a graphics processing unit 448 and an audio processing unit 450, which may be configured to communicate to various external devices such as a display or speakers via one or more A/V ports 452. Example peripheral interfaces 444 include a serial interface controller 454 or a parallel interface controller 456, which may be configured to communicate with external devices such as input devices (e.g., the sensing elements 206 in FIG. 2, keyboard, mouse, pen, voice input device, touch input device, etc.) or other peripheral devices (e.g., printer, scanner, etc.) via one or more I/O ports 458. An example communication device 446 includes a network controller 460, which may be arranged to facilitate communications with one or more other computing devices 462 over a network communication link via one or more communication ports 464.

The network communication link may be one example of communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

The computing device 400 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. The computing device 400 may also be implemented as a personal computer including both laptop computer and non-laptop computer configurations.

From the foregoing, it will be appreciated that specific embodiments of the disclosed technology have been described herein for purposes of illustration, but that various modifications may be made without deviating from the disclosure. Certain aspects of the disclosure described in the context of particular embodiments may be combined or eliminated in other embodiments. Not all embodiments need necessarily exhibit such advantages to fall within the scope of the disclosure. Accordingly, the invention is not limited by the disclosure, but instead its scope is to be determined entirely by the following claims.

I/We claim:

1. A method, comprising:
measuring, using sensing elements individually associated with multiple users, a first set of sleep data associated with the users operating according to a first duty schedule, the sleep data containing at least one or more of sleep starting time, sleep ending time, or time of day during sleep corresponding to each user;
transmitting to and storing in a computing device the measured first set of sleep data;
with the computing device,
applying, a fatigue model to the first set of sleep data to generate a first set of fatigue values;
estimating a second set of sleep data associated with the users according to a second duty schedule different than the first duty schedule without subjecting the individuals to perform duties according to the second duty schedule;
applying the fatigue model to the second set of sleep data to generate a second set of fatigue values; and
determining if the second duty schedule is equivalent to the first duty schedule based on the generated first and second sets of fatigue values.

2. The method of claim 1, further comprising, in response to determining that the second duty schedule is not equivalent or non-inferior to the first duty schedule, estimating a third set of sleep data associated with the one or more individuals according to a third duty schedule different than the first and second duty schedules without subjecting the individuals to perform duties according to the second duty schedule, applying the fatigue model to the third set of sleep data to generate a third set of fatigue values, and determining if the third duty schedule is statistically equivalent to the first duty schedule based on the generated first and third sets of fatigue values.

3. The method of claim 1 wherein both the first and second sets of fatigue values include a distribution of values, and wherein the process performed by the processor further includes calculating one or more statistical parameters for each of the first and second sets of fatigue values.

4. The method of claim 1 wherein:
both the first and second sets of fatigue values include a distribution of values;
the process performed by the processor further includes calculating one or more statistical parameters for each of the first and second sets of fatigue values; and
determining if the second duty schedule is statistically equivalent or non-inferior to the first duty schedule includes determining if the second duty schedule is statistically equivalent or non-inferior to the first duty schedule based on the calculated one or more statistical parameters for each of the first and second sets of fatigue values.

5. A computing system having a processor and a memory containing instructions that when executed by the processor, cause the processor to perform a process comprising:
applying a fatigue model on a first set of sleep data associated with one or more individuals according to a first duty schedule to generate a first set of fatigue values, the sleep data containing at least one or more of sleep starting time, sleep ending time, or time of day during sleep corresponding to each individual;
estimating a second set of sleep data associated with the one or more individuals according to a second duty schedule different than the first duty schedule without subjecting the individuals to perform duties according to the second duty schedule;
applying the fatigue model to the second set of sleep data to generate a second set of fatigue values; and
determining if the second duty schedule is statistically equivalent to the first duty schedule based on the generated first and second sets of fatigue values.

6. The computing system of claim 5 wherein the process performed by the processor further includes, in response to determining that the second duty schedule is not statistically equivalent to the first duty schedule, estimating a third set of sleep data associated with the one or more individuals according to a third duty schedule different than the first and second duty schedules without subjecting the individuals to perform duties according to the second duty schedule, applying the fatigue model to the third set of sleep data to generate a third set of fatigue values, and determining if the third duty schedule is statistically equivalent to the first duty schedule based on the generated first and third sets of fatigue values.

7. The computing system of claim 5 wherein both the first and second sets of fatigue values include a distribution of values, and wherein the process performed by the processor further includes calculating one or more statistical parameters for each of the first and second sets of fatigue values.

8. The computing system of claim 5 wherein:
both the first and second sets of fatigue values include a distribution of values;
the process performed by the processor further includes calculating one or more statistical parameters for each of the first and second sets of fatigue values; and
determining if the second duty schedule is statistically equivalent to the first duty schedule includes determining if the second duty schedule is statistically equivalent to the first duty schedule based on the calculated one or more statistical parameters for each of the first and second sets of fatigue values.

9. The computing system of claim 5 wherein:
both the first and second sets of fatigue values include a distribution of values;
the process performed by the processor further includes calculating a statistical parameter for each of the first and second sets of fatigue values, the statistical parameter including one of a mean, an average, a range, one or more quantile values, a standard deviation, a mean difference, a median absolute deviation, an average absolute deviation, or a distance standard deviation; and
determining if the second duty schedule is statistically equivalent to the first duty schedule includes determining if the second duty schedule is statistically equivalent to the first duty schedule based on the calculated statistical parameter for each of the first and second sets of fatigue values.

10. The computing system of claim 5 wherein:
both the first and second sets of fatigue values include a distribution of values;
the process performed by the processor further includes calculating a statistical parameter for each of the first and second sets of fatigue values, the statistical parameter including one of a mean, an average, a range, one or more quantile values, a standard deviation, a mean difference, a median absolute deviation, an average absolute deviation, or a distance standard deviation;
determining if the second duty schedule is statistically equivalent to the first duty schedule includes determining if the second duty schedule is statistically equivalent to the first duty schedule based on the calculated statistical parameter for each of the first and second sets of fatigue values; and
the process performed by the processor further includes indicating that the second duty schedule is statistically equivalent to the first duty schedule in response to determining that a difference between the calculated statistical parameters is within a threshold.

11. A method performed by a computing device having a processor, comprising:
with the processor,
receiving a first set of sleep data associated with one or more individuals operating according to a first duty schedule, the sleep data containing at least one or more of sleep starting time, sleep ending time, or time of day during sleep corresponding to each individual;
receiving a second set of sleep data associated with the one or more individuals operating according to a second duty schedule different than the first duty schedule;
generating first and second sets of fatigue values based on the first and second sets of sleep data, respectively; and
determining a statistical relationship of fatigue risk between the first duty schedule and the second duty schedule based on the generated first and second sets of fatigue values.

12. The method of claim 11 wherein at least one of receiving the first set of sleep data or receiving the second set of sleep data includes receiving the first set of sleep data from a rest/activity monitor associated with each individual on a periodic or continuous basis.

13. The method of claim 11 wherein receiving the second set of sleep data includes estimating the second set of sleep data based at least on a circadian parameter corresponding to each individual or estimating the second set of sleep data by recording the sleep data of the individuals without the individuals actually performing duties.

14. The method of claim 11 wherein receiving the first set of sleep data and receiving the second set of sleep data include estimating the first set and second set of the sleep data, respectively.

15. The method of claim 11 wherein receiving the second set of sleep data includes estimating the second set of sleep data by obtaining sleep data from areas of operation other than that associated with the individuals or by interpolating, extrapolating, or imputing from the first set of sleep data.

16. The method of claim 11 wherein at least one of receiving the first set of sleep data or receiving the second set of sleep data includes sampling from a set of sleep data associated with the first or second schedule, respectively.

17. The method of claim 11 wherein determining the statistical relationship includes calculating a statistical parameter for each of the generated first and second sets of fatigue values, the statistical parameter including one of a mean, an average, a range, one or more quantile values, a standard deviation, a mean difference, a median absolute deviation, an average absolute deviation, or a distance standard deviation.

18. The method of claim 11 wherein determining the statistical relationship includes calculating a statistical parameter for each of the generated first and second sets of fatigue values and comparing the calculated statistical parameters corresponding to the generated first and second sets of fatigue values.

19. The method of claim 11 wherein:
determining the statistical relationship includes calculating a statistical parameter for each of the generated first and second sets of fatigue values, the statistical parameter including one of a mean, an average, a range, one or more quantile values, a standard deviation, a mean difference, a median absolute deviation, an average absolute deviation, or a distance standard deviation;
determining the statistical relationship further includes:
comparing the calculated statistical parameters corresponding to the generated first and second sets of fatigue values; and
indicating that the second duty schedule is statistically non-inferior if the calculated statistical parameter of the generated second set of fatigue values is greater than or equal to that of the first set of fatigue values.

20. The method of claim 11 wherein:
determining the statistical relationship includes calculating a statistical parameter for each of the generated first and second sets of fatigue values, the statistical parameter including one of a mean, an average, a range, one or more quantile values, a standard deviation, a mean difference, a median absolute deviation, an average absolute deviation, or a distance standard deviation;
determining the statistical relationship further includes:
comparing the calculated statistical parameters corresponding to the generated first and second sets of fatigue values;
in response to that the calculated statistical parameter of the generated second set of fatigue values is greater than or equal to that of the first set of fatigue values, indicating that the second duty schedule is statistically non-inferior than the first duty schedule; and
in response to that the calculated statistical parameter of the generated second set of fatigue values is less than that of the first set of fatigue values,
estimating a third set of sleep data associated with the one or more individuals operating according to a third duty schedule different than the first or second duty schedule; and
repeating the generating and determining operations.
